(19) United States
(12) Patent Application Publication
(10) Pub. No.: US 2009/0226508 A1
(43) Pub. Date: Sep. 10, 2009

(54) Methods And Materials For Immunization Against Cancer

(76) Inventors: John W. Eaton, Prospect, KY (US); Robert A. Mitcheli, Louisville, KY (US)

Correspondence Address: JENKINS, WILSON, TAYLOR & HUNT, P. A., Suite 1200 UNIVERSITY TOWER, 3100 TOWER BLVD.,, DURHAM, NC, 27707, US

(21) Appl. No.: 12/225,140

(22) PCT Filed: Mar. 15, 2007
(86) PCT No.: PCT/US07/06578

§ 371 Date: May 5, 2009

Related U.S. Application Data
(60) Provisional application No. 60/782,672, filed on Mar. 15, 2006.

(51) Int. Cl. A61K 35/12; A61K 9/127; A61K 39/395; A61K 39/00; A61K 38/20; A61K 38/21; G01N 33/53; C40B 30/04; A61P 35/00

(52) U.S. Cl. 424/450; 424/93.7; 424/130.1; 424/184.1; 424/85.2; 424/85.7; 424/85.5; 435/7.1; 506/9

(57) ABSTRACT

Disclosed are compositions comprising embryonic stem cells. In some embodiments, the compositions include a plurality of pluripotent cells and one or more pharmaceutically acceptable carriers or excipients. Also disclosed are methods for pro-phylaxis and/or treatment of subjects using the disclosed compositions, and methods for identifying an antigen shared by a pluripotent cell and a neoplastic or pre-neoplastic cell.

### CROSS REFERENCE TO RELATED APPLICATION

This application is based on and claims priority to United States of America Provisional Patent Application Ser. No. 60/782,672, filed Mar. 15, 2006, the disclosure of which is herein incorporated by reference in its entirety.

### TECHNICAL FIELD

The presently disclosed subject matter relates, in general, to compositions and methods for prevention of and/or treatment of a tumor and/or a cancer. More particularly, the presently disclosed subject matter relates to administering prophylactic and/or therapeutic compositions comprising pluripotent cells to a subject in need thereof. Also provided are methods for identifying antigens shared by pluripotent cells and neoplastic or pre-neoplastic cells.

### BACKGROUND

Most currently available immunotherapies for malignancies using vaccination involve attempts to provoke active immunity to individual antigens. Unfortunately, these approaches have met with limited success. An alternative strategy could conceivably involve prophylactic vaccination against tumor antigens prior to the appearance of cancer, but this approach also faces challenges. Unlike infectious cancer-causing agents for which effective vaccines have been produced, spontaneous mutation-initiated tumor cells derive from “self”. Consequently, the majority of antigens displayed by tumor cells can also be present on normal adult cells and therefore can be non-immunogenic. If, however, a source of antigens presented by a variety of tumors—but not by adult tissues—were available, the prospects for creating a broad-spectrum cancer vaccine could be vastly improved.

Thus, there continues to be a need for new approaches to prevent and/or treat neoplastic disease. This and other needs are addressed in whole or in part by the presently disclosed subject matter.

### SUMMARY

This Summary lists several embodiments of the presently disclosed subject matter, and in many cases lists variations and permutations of these embodiments. This Summary is merely exemplary of the numerous and varied embodiments. Mention of one or more representative features of a given embodiment is likewise exemplary. Such an embodiment can typically exist with or without the feature(s) mentioned; likewise, those features can be applied to other embodiments of the presently disclosed subject matter, whether listed in this Summary or not. To avoid excessive repetition, this Summary does not list or suggest all possible combinations of such features.

The presently disclosed subject matter provides compositions and methods for prevention and/or treatment of a tumor and/or a cancer.

In some embodiments, the presently disclosed subject matter provides methods for suppressing growth of a cancer in a subject. In some embodiments, the methods comprise administering to the subject a composition comprising a plurality of pluripotent cells and one or more pharmaceutically acceptable carriers or excipients, whereby growth of the cancer in the subject is suppressed. In some embodiments, the pluripotent cells are mammalian pluripotent cells. In some embodiments, the plurality of pluripotent cells comprise embryonic stem cells including, but not limited to human embryonic stem cells, mouse embryonic stem cells, or combinations thereof. In some embodiments, the pluripotent cells are allogeneic or xenogeneic to the subject. In some embodiments, the pluripotent cells are present in the composition in an amount ranging from about 1×10⁵ to about 1×10⁷ pluripotent cells per dose.

In some embodiments of the presently disclosed subject matter, the pluripotent cells have been manipulated prior to administration to the subject. In some embodiments, the presently disclosed methods further comprise exposing the plurality of pluripotent cells to a treatment under conditions sufficient to prevent the pluripotent cells from forming a tumor in the subject. In some embodiments, the exposing step comprises exposing the plurality of pluripotent cells to at least one chemical, to radiation, or to combinations thereof.

The presently disclosed subject matter also provides methods for inducing an anti-tumor immune response in a subject. In some embodiments, the methods comprise administering to the subject a composition comprising a plurality of pluripotent cells and one or more pharmaceutically acceptable carriers or excipients. In some embodiments, the anti-tumor immune response is sufficient to (a) prevent occurrence of a tumor in the subject; (b) delay occurrence of a tumor in the subject; (c) reduce a rate at which a tumor develops in the subject; (d) prevent recurrence of a tumor in the subject; (e) suppress growth of a tumor in a subject; or (f) combinations thereof. In some embodiments, the anti-tumor immune response comprises a cytotoxic T cell response against an antigen present in or on a cell of the tumor. In some embodiments, the cytotoxic T cell response is mediated by CD8+ T cells.

The presently disclosed subject matter also provides compositions for use in the disclosed methods. In some embodiments, the compositions comprise a plurality of pluripotent cells and one or more pharmaceutically acceptable carriers or excipients, wherein the plurality of pluripotent cells are inactivated to an extent sufficient to prevent the pluripotent cells from forming a tumor when administered to a subject. In some embodiments, the compositions are in the form of a vaccine. In some embodiments, the pluripotent cells are mammalian pluripotent cells. In some embodiments, the mammalian pluripotent cells comprise human embryonic stem cells, mouse embryonic stem cells, or a combination thereof. In some embodiments, the pluripotent cells are present in the composition in an amount ranging from about 1×10⁵ to about 1×10⁷ cells per dose. In some embodiments of the presently disclosed compositions, the pluripotent cells are present in the compositions in a form selected from the group consisting of whole cells, fractions of cells, cell lysates, and combinations thereof. In some embodiments, the one or more pharmaceutically acceptable carriers or excipients are pharmaceutically acceptable for use in a human.

The presently disclosed compositions can optionally include additional biologically active components. In some embodiments, the compositions further comprise a biologically active component comprising an adjuvant, a biological response modifier, or a combination thereof. In some embodiments, the biological response modifier is selected from the group consisting of a cytokine and an anti-tolerance drug. In some embodiments, the biological response modifier comprises a cytokine selected from the group consisting of an interferon alpha (IFN-α), an interferon gamma (IFN-γ), an interleukin 2 (IL-2), an interleukin 4 (IL-4), an interleukin 6 (IL-6), an interleukin 12 (IL-12), a tumor necrosis factor (TNF), and a granulocyte-macrophage colony stimulating factor (GM-CSF), functional fragments thereof, and combinations thereof. In some embodiments, the biological response modifier comprises a granulocyte-macrophage colony stimulating factor (GM-CSF), a functional fragment thereof, or a source thereof. In some embodiments, the GM-CSF or functional fragment thereof is present in the composition in a form selected from the group consisting of a GM-CSF polypeptide, a cell expressing a GM-CSF polypeptide, and an encapsulated GM-CSF polypeptide. In some embodiments, the functional GM-CSF polypeptide is encapsulated in a microsphere or a liposome. In some embodiments, the functional GM-CSF comprises a human GM-CSF polypeptide. In some embodiments, the anti-tolerance drug is selected from the group consisting of an anti-CTLA4 antibody and ONTAK® (denileukin diftitox).

The presently disclosed compositions can be administered to a subject in need thereof at any time in which the induction of an anti-tumor immune response would be desirable. In some embodiments, the administering is performed prior to a diagnosis of a presence of a tumor and/or a cancer in the subject, and the composition is in the form of a vaccine. In some embodiments, the subject in need thereof has at least one risk factor for development of the cancer. In some embodiments, the risk factor comprises a genetic or chronologic predisposition for development of the cancer. In some embodiments, the risk factor comprises exposure to a carcinogen. In some embodiments, the exposure comprises use of a tobacco product. In some embodiments, the administering step is performed at least twice.

The presently disclosed compositions and methods can also be employed as part of a multi-component anti-tumor and/or anti-cancer treatment modality. In some embodiments, the presently disclosed methods further comprise providing to the subject an additional anti-cancer therapy selected from the group consisting of radiation, chemotherapy, surgical resection, immunotherapy, and combinations thereof. In some embodiments, the additional anti-cancer therapy is provided to the subject at a time prior to, concurrent with, subsequent to, or combinations thereof, the administering step. In some embodiments, the additional anti-cancer therapy is provided prior to the administering step and the composition is administered as an adjuvant therapy.

The presently disclosed compositions and methods can be employed for the prevention and/or treatment of any tumor and/or any cancer. In some embodiments, the cancer is selected from the group consisting of bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor. In some embodiments, the cancer comprises a lung carcinoma.

The presently disclosed compositions and methods can be employed for prevention and/or treatment of a tumor and/or a cancer in any subject. In some embodiments, the subject is a mammal. In some embodiments, the mammal is a human.

The presently disclosed subject matter also provides methods for identifying an antigen shared by a pluripotent cell and a neoplastic or pre-neoplastic cell. In some embodiments, the methods comprise (a) administering to a first subject a plurality of pluripotent cells to induce an immune response in the subject against one or more antigens present on one or more of the pluripotent cells; (b) isolating an antiserum, a lymphocyte, a splenocyte, or a combination thereof from the immunized subject; (c) assaying the antiserum, lymphocyte, splenocyte, or combination thereof for an ability to recognize an antigen present on a neoplastic or pre-neoplastic cell; and (d) identifying the antigen present on the neoplastic or pre-neoplastic cell to which the antiserum, lymphocyte, splenocyte, or combination thereof binds. In some embodiments, the antigen present on the neoplastic or pre-neoplastic cell is provided in the assaying step as a constituent of a library. In some embodiments, the library comprises an expression library produced from a neoplastic or pre-neoplastic cell. In some embodiments, the expression library is produced from a neoplastic or pre-neoplastic cell isolated from a second subject, optionally further wherein the first subject and the second subject are the same individual. In some embodiments, the neoplastic or pre-neoplastic cell from which the library is made is isolated from the second subject as part of a biopsy or resection. In some embodiments, the plurality of pluripotent cells are allogeneic or xenogeneic to the first subject, the second subject, or both the first and the second subjects. In some embodiments, the presently disclosed methods further comprise removing from the antiserum one or more antibodies that bind to a control tissue in the first subject, the second subject, or both the first and the second subjects.

Accordingly, it is an object of the presently disclosed subject matter to provide compositions comprising pluripotent cells. This and other objects are achieved in whole or in part by the presently disclosed subject matter.

An object of the presently disclosed subject matter having been stated above, other objects and advantages of the presently disclosed subject matter will become apparent to those of ordinary skill in the art after a study of the following description, non-limiting Examples, and Figures.

### BRIEF DESCRIPTION OF THE DRAWINGS

FIGS. 1A-1C are bar graphs presenting the results of embryonic stem (ES) cell vaccination on 3-methylcholanthrene initiated, butylated hydroxytoluene promoted lung carcinogenesis. C57BL/6 mice were treated with 3-methylcholanthrene and repetitive injection of butylated hydroxytoluene Mice were vaccinated with murine ES cells alone or murine ES cells+GM-CSF-expressing STO fibroblasts. Mice were sacrificed at week 18, lungs dissected, and 3 sets of randomly selected lungs from each group were sectioned and stained. Adenomatous lesions (adenomas and adenocarcinomas) throughout the lung were quantified from digitized micrographs (Nikon COOL PIX® camera), and the results are presented as the total number of lesions (FIG. 1A), average cross-sectional area of lesions (FIG. 1B), and percentage of total lung area comprised of lesions (adenomas+adenocarcinomas; FIG. 1C).

FIG. 2 depicts a Western blot of lysates of COS-7 cells transfected with either an empty vector (COS-7 vector) or a cytohesin-2 expression vector (COS-7 Cytohesin-2) 48 hours after transfection. Cell lysates from the indicated cells were normalized for protein content and subjected to SDS-PAGE and immunoblotted for murine cytohesin-2 using a commercially available monoclonal antibody (mAb; Sigma, St. Louis, Mo., United States of America). Also included are lysates from murine ES cells (ES) and LLC cells (LLC). β-actin was included as a loading control for each lane and was detected after stripping the blots that had been probed with the anti-cytohesin-2 mAb.

FIG. 3 is a bar graph showing that cytohesin-2 induced activation/proliferation of splenocytes from murine embryonic stem cell (ESC)-vaccinated mice. COS-7 cells were transfected with either an empty vector or a cytohesin-2 expression vector. After 48 hours, cell lysates were produced in sterile PBS from the indicated cells and were incubated (15 μg/well) with either 5×10⁵ splenocytes/well (in triplicate) from control (hatched bar) or ESC vaccinated (3×) mice (solid bars). Control bars show PBS alone. 72 hours after antigen was addition, 1 μCi of ³H-thymidine was added to each well and incubated for an additional 16 hours.

FIG. 4 is a graph showing the percentage of human ES-cell-immunized mice that were tumor free at various times after challenge with 5×10⁴ Lewis Lung Carcinoma (LLC) cells. Groups of 10 mice were vaccinated on day −14 with 1×10⁶ BG02 human ES cells (open circles) or 1×10⁶ BG02 human ES cells that had been irradiated with 10 Gray (open triangles). At day −7, each group of mice was boosted with a dosage equivalent to that they received on day −14. Day 0 corresponds to the day on which the LLC cells were implanted. A set of control mice that were not vaccinated was also analyzed (solid square).

### DETAILED DESCRIPTION

The presently disclosed subject matter relates, in general, to compositions and methods for prevention of and/or treatment of a tumor and/or a cancer. More particularly, the presently disclosed subject matter relates to administering prophylactic and/or therapeutic compositions comprising pluripotent cells to a subject in need thereof. Also provided are methods for identifying antigens shared by pluripotent cells and neoplastic or pre-neoplastic cells.

### I. DEFINITIONS

While the following terms are believed to be well understood by one of ordinary skill in the art, the following definitions are set forth to facilitate explanation of the presently disclosed subject matter.

All technical and scientific terms used herein, unless otherwise defined below, are intended to have the same meaning as commonly understood by one of ordinary skill in the art. References to techniques employed herein are intended to refer to the techniques as commonly understood in the art, including variations on those techniques or substitutions of equivalent techniques that would be apparent to one of skill in the art. While the following terms are believed to be well understood by one of ordinary skill in the art, the following definitions are set forth to facilitate explanation of the presently disclosed subject matter.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in this specification and attached claims are approximations that can vary depending upon the desired properties sought to be obtained by the presently disclosed subject matter.

Following long-standing patent law tradition, the terms “a”, “an”, and “the” are meant to refer to one or more as used herein, including the claims. For example, the phrase “a cell” can refer to one or more cells. Also as used herein, the term “another” can refer to at least a second or more.

The term “about”, as used herein when referring to a measurable value such as an amount of weight, time, dose (e.g., a number of cells), etc., is meant to encompass variations of in some embodiments ±20%, in some embodiments ±10%, in some embodiments, ±5%, in some embodiments ±1%, and in some embodiments ±0.1% from the specified amount, as such variations are appropriate to perform the disclosed methods.

The use of the term “or” in the claims is used to mean “and/or” unless explicitly indicated to refer to alternatives only or the alternatives are mutually exclusive.

As used herein, the words “comprising” (and any form of comprising, such as “comprise” and “comprises”), “having” (and any form of having, such as “have” and “has”), “including” (and any form of including, such as “includes” and “include”), or “containing” (and any form of containing, such as “contains” and “contain”) are inclusive or open-ended and do not exclude additional, unrecited elements or method steps.

As used herein, the terms “nucleic acid” and “nucleic acid molecule” mean any of deoxyribonucleic acid (DNA), ribonucleic acid (RNA), oligonucleotides, fragments generated by the polymerase chain reaction (PCR), and fragments generated by any of ligation, scission, endonuclease action, and exonuclease action. Nucleic acids can be composed of monomers that are naturally occurring nucleotides (such as deoxyribonucleotides and ribonucleotides), or analogs of naturally occurring nucleotides (e.g., α-enantiomeric forms of naturally-occurring nucleotides), or a combination of both. Nucleic acids can be either single stranded or double stranded.

As used herein, the term “polypeptide” means any polymer comprising any of the 20 protein amino acids, or amino acid analogs, regardless of its size or function. Although “protein” is often used in reference to relatively large polypeptides, and “peptide” is often used in reference to small polypeptides, usage of these terms in the art overlaps and varies. The term “polypeptide” as used herein refers to peptides, polypeptides and proteins, unless otherwise noted. As used herein, the terms “protein”, “polypeptide” and “peptide” are used interchangeably. The term “polypeptide” encompasses proteins of all functions, including enzymes.

As used herein, the term “GM-CSF polypeptide” refers to a full length granulocyte-macrophage colony stimulating factor (also referred to as colony stimulating factor 2) polypeptide as well as to fragments thereof that have at least a fraction of an immunomodulatory activity of the full length polypeptide.

As used herein, “significance” or “significant” relates to a statistical analysis of the probability that there is a non-random association between two or more occurrences. To determine whether or not a relationship is “significant” or has “significance”, statistical manipulations of the data can be performed to calculate a probability, expressed as a “p-value”. Those p-values that fall below a user-defined cutoff point are regarded as significant. In some embodiments, a p-value less than or equal to 0.10, in some embodiments less than or equal to 0.05, in some embodiments less than or equal to 0.01, in some embodiments less than or equal to 0.005, and in some embodiments less than or equal to 0.001, are regarded as significant.

The term “subject” as used herein refers to a member of any invertebrate or vertebrate species. The methods of the presently disclosed subject matter are particularly useful for warm-blooded vertebrates. Thus, the presently disclosed subject matter concerns mammals and birds. Provided in some embodiments is the treatment and/or prophylaxis of tumors in mammals such as humans, as well as those mammals of importance due to being endangered (such as Siberian tigers), of economic importance (animals raised on farms for consumption by humans) and/or social importance (animals kept as pets or in zoos) to humans, for instance, carnivores other than humans (such as cats and dogs), swine (pigs, hogs, and wild boars), ruminants (such as cattle, oxen, sheep, giraffes, deer, goats, bison, and camels), and horses. Also provided is the use of the disclosed methods and compositions on birds, including those kinds of birds that are endangered, kept in zoos, as well as fowl, and more particularly domesticated fowl, e.g., poultry, such as turkeys, chickens, ducks, geese, guinea fowl, and the like, as they are also of economic importance to humans. Thus, provided is the use of the disclosed methods and compositions in livestock, including but not limited to domesticated swine (pigs and hogs), ruminants, horses, poultry, and the like.

As used herein, the term “syngeneic” refers to two or more organisms of the same species, or tissues and/or cells therefrom, that are genetically identical or so closely related genetically that transplantation of cells and/or tissues between the two or more organisms is tolerated without the need for immune system suppression to prevent graft-versus-host disease or host-versus-graft disease. Stated another way, two organisms of the same species are said to be syngeneic if their cells and/or tissues are histocompatible, wherein histocompatible refers to the fact that the transplantation and/or grafting of a tissue from one immunocompetent individual to another immunocompetent individual does not result in rejection of the transplant/graft and does not require the use of immunosuppressive therapy.

As such, “syngeneic” is to be contrasted with “allogeneic”, the latter of which refers to two or more organisms of the same species, or tissues and/or cells therefrom, that are genetically distinct at least to the extent that transplantation of cells and/or tissues between the two or more organisms is not tolerated and would result in rejection of the transplant in the absence of immunosuppression. It is understood, therefore, that two organisms of the same species can be allogeneic even when there is some degree of overlap between histocompatibility antigens, provided that the immune system of one of the organisms would be capable of mounting an immune response against a transplanted cell or tissue from the organism (e.g., would recognize at least one antigen present on and/or in the transplanted cell and/or tissue as non-self). Thus, for example, C57BL/6 mice and 129/Sv mice are considered allogeneic for the purposes of the presently disclosed subject matter despite the fact that each is characterized by an H2-b histocompatibility haplotype. Additionally, 129/Sv mice, or cells derived therefrom (e.g., the D3 ES cell line), and BALB/c mice, or cells therefrom, are allogeneic as the former is H2-b and the latter is H2-d.

The terms “syngeneic” and “allogeneic” are also to be contrasted with the term “xenogeneic”, which refers to two or more organisms of different species, or tissues and/or cells therefrom. In some embodiments, xenogeneic pluripotent cells are employed in the compositions of the presently disclosed subject matter, meaning that the pluripotent cells that are present in the compositions are intended for administration to a subject of a species other than the species from which the pluripotent cells were isolated or derived.

As used herein, the term “teratoma” refers to a tumor characterized by the unregulated development of ES cells after transfer into a subject. Teratomas often generate cells of several different cell and tissue types, such as of skin, hair, cartilage, and muscle. ES cells injected into syngeneic mice can form teratomas that exhibit disorganized differentiation, often with representatives of all three embryonic germ layers.

As used herein, the phrases “treatment effective amount”, “therapeutically effective amount”, “treatment amount”, and “effective amount” are used interchangeably and refer to an amount of a composition (e.g., a plurality of ES cells and/or other pluripotent cells in a pharmaceutically acceptable carrier or excipient) sufficient to produce a measurable response (e.g., a biologically or clinically relevant response in a subject being treated). For example, actual dosage levels of ES cells and/or other pluripotent cells in the compositions of the presently disclosed subject matter can be varied so as to administer a sufficient number of ES cells and/or other pluripotent cells to achieve the desired immune response for a particular subject. The selected dosage level will depend upon several factors including, but not limited to the route of administration, combination with other drugs or treatments, the severity of the condition being treated, and the condition and prior medical history of the subject being treated.

Additionally, in the context of the prophylactic methods disclosed herein, a phrases “treatment effective amount”, “therapeutically effective amount”, “treatment amount”, and “effective amount” refer to an amount that elicits an immune response sufficient to provide a prophylactic benefit to the subject. In some embodiments, a prophylactic benefit is provided by inducing an immune response to an antigen and/or epitope present in the composition sufficient to prevent the initial occurrence and/or growth of a tumor and/or a cancer, delay the occurrence and/or growth of a tumor and/or a cancer in the subject, reduce a rate at which a tumor develops in the subject; or combinations thereof.

The terms “cancer” and “tumor” are used interchangeably herein and can refer to both primary and metastasized solid tumors and carcinomas of any tissue in a subject, including but not limited to breast; colon; rectum; lung; oropharynx; hypopharynx; esophagus; stomach; pancreas; liver; gallbladder; bile ducts; small intestine; urinary tract including kidney, bladder, and urothelium; female genital tract including cervix, uterus, ovaries (e.g., choriocarcinoma and gestational trophoblastic disease); male genital tract including prostate, seminal vesicles, testes and germ cell tumors; endocrine glands including thyroid, adrenal, and pituitary; skin (e.g., hemangiomas and melanomas), bone or soft tissues; blood vessels (e.g., Kaposi's sarcoma); brain, nerves, eyes, and meninges (e.g., astrocytomas, gliomas, glioblastomas, retinoblastomas, neuromas, neuroblastomas, Schwannomas and meningiomas). The terms “cancer and “tumor” also encompass solid tumors arising from hematopoietic malignancies such as leukemias, including chloromas, plasmacytomas, plaques and tumors of mycosis fungoides and cutaneous T-cell lymphoma/leukemia, and lymphomas including both Hodgkin's and non-Hodgkin's lymphomas. As used herein, the terms “cancer and “tumor” are also intended to refer to multicellular tumors as well as individual neoplastic or pre-neoplastic cells. In some embodiments, a tumor is an adenoma and/or an adenocarcinoma, in some embodiments a lung adenoma and/or adenocarcinoma.

### II. COMPOSITIONS

### II.A. Pluripotent Cells and Derivatives Thereof

The compositions of the presently disclosed subject matter comprise one or more antigens expressed by pluripotent cells. In general, such antigens can include any epitope expressed by pluripotent cells including, but not limited to those epitopes that are present on the surface and/or the membrane of pluripotent cells and/or are secreted or released by pluripotent cells.

In some embodiments, the antigens are administered to a subject in a composition comprising a population of cells, e.g., pluripotent cells or cells having one or more antigens expressed by pluripotent cells. In some embodiments, the antigens are carried by one or more subcellular fractions, e.g., cell lysates, cell membrane fractions, cell plasma fractions, cell protein fractions, etc. In some embodiments, the antigens comprise epitopes expressed by pluripotent cells as well as by neoplastic (e.g., tumor or cancerous) or pre-neoplastic cells. For example, the antigens can comprise any epitope derived from cytohesin-2.

According to the presently disclosed subject matter, these compositions are suitable for vaccine formulations and/or therapeutic formulations. In some embodiments, the composition of the presently disclosed subject matter does not contain substantial amount of cell growth medium (e.g., does not include pluripotent cells maintained in a cell culture). In some embodiments, the composition of the presently disclosed subject matter is a vaccine formulation (e.g., is suitable for the treatment of a human). In some embodiments, the composition of the presently disclosed subject matter is a therapeutic formulation (e.g., suitable for the treatment of a human).

In some embodiments of the presently disclosed subject matter, pluripotent cells and/or derivatives thereof are administered to a subject in order to induce an immune response against one or more antigens present on or in the pluripotent cells and/or derivatives thereof. As used herein, the term “pluripotent” refers to a cell that is capable of differentiating into one of several different (although sometimes related) cell types. Pluripotent cells can thus be more restricted in their differentiative capacity than “totipotent” cells, which can differentiate into any and all cell types. As such, the phrase “totipotent” encompasses “pluripotent” although the reverse may not be true.

An exemplary pluripotent cell is a mouse embryonic stem (ES) cell. Mouse ES cells are undifferentiated, pluripotent cells typically derived in vitro from early embryos (Evans et al., 1981; Martin, 1981). Mouse ES cells can maintain an undifferentiated state through serial passages using culturing techniques that are known in the art (see e.g., Robertson et al., 1987; Williams et al., 1988; Nagy et al., 1990; Nagy et al., 2003). In some embodiments, mouse ES cells are cultured on a fibroblast feeder layer and/or in the presence of Leukemia Inhibitory Factor (LIF) to maintain an undifferentiated state.

The cells of a feeder layer are typically mitotically inactivated with mitomycin C or gamma irradiation. An exemplary fibroblast cell that can be used to produce a feeder layer is the STO cell (ATCC® No. CRL-1503™, American Type Culture Collection (ATCCO®), Manassas, Va., United States of America). Additionally, some feeder cells are available that have been modified to express LIF and/or a neomycin resistance gene (neo), the latter of which can be employed to grow ES cells and ES cell derivatives that have been transformed with an expression vector encoding a neomycin phosphotransferase (neo) coding sequence. In some embodiments, if a LIF-producing feeder cell is employed, the use of additional LIF in the ES cell propagation medium can be avoided. STO derivatives are available that have been modified to express both LIF and neo, such as the SNL76/7 fibroblast line described in McMahon & Bradley, 1990, and available from Dr. Allan Bradley, Baylor College of Medicine, Houston, Tex., United States of America. Other STO cell lines that have been modified to express both LIF and neo are available from Dr. Elizabeth Robertson of Harvard University, Cambridge, Mass., United States of America.

Alternatively or in addition, ES cells can be grown on a monolayer of murine embryonic fibroblasts (MEFs) that have been prepared as described in, for example, Loo & Costman, 1998. MEFs can also be prepared from a mouse embryo that has been genetically altered to express a selectable marker (see e.g., Tucker et al., 1997, describing a mouse that expresses resistance genes to G418, 6-thioguanine, puromycin, and hygromycin), which can aid in the propagation of ES cells and ES cell derivatives that have been transformed with recombinant vectors.

In some embodiments including, for example, when the ES cells are intended for use in producing a vaccine for administration into humans, the presence of a feeder layer comprising cells from a species other than humans is disfavored. U.S. Pat. No. 6,800,480 to Bodnar et al. and U.S. Patent Application Publication No. 20060030042 of Brivanlou et al. disclose methods and materials for the growth of stem cells in a feeder-free culture. Thus, in some embodiments, the ES cells are maintained in culture in the absence of a feeder layer and maintained in an undifferentiated state by the addition of exogenous growth factors including, but not limited to LIF. It is understood that any cell culture technique including, but not limited to feeder-free culture and serum-free culture, can be employed in the culture of the pluripotent cells of the presently disclosed subject matter.

For example, when the ES cells are intended for use in producing a vaccine for administration into humans, the growth of the ES cells in animal serum (e.g., bovine serum) is disfavored. U.S. Patent Application Publication No. 20050266553 to Pebav & Pera discloses a method and materials for the growth of stem cells in a serum-free culture. Thus, in some embodiments, the ES cells are maintained in a culture medium absent animal serum and maintained in an undifferentiated state. Exemplary culture conditions for human ES cells are disclosed in EXAMPLE 6 below.

Pluripotent cells (e.g., ES cells) from any species can be employed in the compositions and methods disclosed herein. Additionally, the pluripotent cells need not be from the same species as the subject into which the compositions of the presently disclosed subject matter are administered. Thus, in some embodiments, allogeneic pluripotent cells (i.e., from the same species as the subject) are employed, and in some embodiments xenogeneic pluripotent cells (i.e., from a different species than the subject) are employed. Murine ES cell lines are commercially available (e.g., from the American Type Culture Collection, Manassas, Va., United States of America), and ES cells from other species including humans and other primates (see e.g., U.S. Pat. Nos. 5,843,780; 6,200,806; 6,875,607; and 6,921,632; and Thomson et al., 1995, 1996), birds (see e.g., U.S. Pat. Nos. 5,340,740; 5,656,479; and 5,830,510), and pigs (Li et al., 2004) have also been produced. In some embodiments, the compositions disclosed herein comprise human pluripotent cells.

It is understood that a function of the pluripotent cells, derivatives thereof, and/or fractions thereof disclosed herein is to provide one or more antigens that are shared between the pluripotent cells and a cancer cell to a subject to which they are administered. Thus, the pluripotent cell's status as being pluripotent is not determinative of the cell's usefulness in the presently disclosed methods and compositions. Indeed, other cells and cell types can be employed in the disclosed methods and compositions. For example, such cells and cell types include, but are not limited to early primitive ectoderm-like (EPL) cells as described in PCT International Patent Application Publication WO 99/53021; in vivo or in vitro derived inner cell mast/epiblast; in vivo or in vitro derived primitive ectoderm; primordial germ cells (PGCs), including embryonic germ (EG) cells derived therefrom; teratocarcinoma cells (EC cells), and cells derived by dedifferentiation or by nuclear transfer.

With respect to EG cells, these cells are ES-like cells that can be generated from primordial germ cells (PGCs) from several different species including, but not limited to mice (see U.S. Pat. Nos. 5,453,357; 5,670,372; 5,690,926; all to Hogan), pigs (see U.S. Pat. No. 6,271,436 to Piedrahita & Bazer), bovines (U.S. Pat. No. 6,011,197 to Strelchenko et al.), avians (U.S. Pat. No. 6,333,192 to Petitte & Chan), and humans (see U.S. Pat. Nos. 6,090,622; 6,245,566; and 6,331,406; all to Gearhart & Shamblott). See also Shamblott et al., 1998. These cells are also intended to be encompassed by the phrase “pluripotent cells” as that phrase is used herein.

In some embodiments, the pluripotent cells are inactivated. In some embodiments, the term “inactivated”, and grammatical variants thereof, is used herein to refer to a cell (e.g., a pluripotent cell) that is alive but has been rendered incapable of proliferation (i.e., mitotically inactivated). Using techniques that are known in the art including, but not limited to exposure to chemical agents and/or irradiation, pluripotent cells can be inactivated such that upon administration to a subject the pluripotent cells are incapable of dividing and thus cannot form teratomas in the subject. It is understood that in the context of a plurality of cells, not every cell needs to be incapable of proliferation. Thus, as used herein the phrase “inactivated to an extent sufficient to prevent teratoma formation in the subject” refers to a degree of inactivation in the population as a whole such that after administration to a subject a teratoma does not form even if a one or more cells in the plurality of cells are in fact capable of proliferation in the subject. While applicants do not wish to be bound by any particular theory of operation, it is possible that even if some cells are capable of proliferating, the subject's immune system would eliminate these cells before a teratoma could form. In some embodiments, an “inactivated” cell is a killed cell, and in some embodiments, the inactivated cell is a fraction of a cellular lysate such as a membrane fraction, a cytoplasmic fraction, or a combination thereof.

### II.B. Formulation

The compositions of the presently disclosed subject matter comprise in some embodiments a pharmaceutically acceptable carrier. Any suitable formulation can be used to prepare the disclosed compositions for administration to a subject. In some embodiments, the pharmaceutically acceptable carrier is pharmaceutically acceptable for use in a human.

For example, suitable formulations can include aqueous and non-aqueous sterile injection solutions which can contain anti-oxidants, buffers, bacteriostats, bactericidal antibiotics and solutes which render the formulation isotonic with the bodily fluids of the intended recipient; and aqueous and non-aqueous sterile suspensions which can include suspending agents and thickening agents. The formulations can be presented in unit-dose or multi-dose containers, for example sealed ampoules and vials, and can be stored in a frozen or freeze-dried (lyophilized) condition requiring only the addition of sterile liquid carrier, for example water for injections, immediately prior to use. Some exemplary ingredients are SDS, in some embodiments in the range of 0.1 to 10 mg/ml, in some embodiments about 2.0 mg/ml; and/or mannitol or another sugar, in some embodiments in the range of 10 to 100 mg/ml and in some embodiments about 30 mg/ml; and/or phosphate-buffered saline (PBS).

It should be understood that in addition to the ingredients particularly mentioned above, the formulations of the presently disclosed subject matter can include other agents conventional in the art having regard to the type of formulation in question. Of the possible formulations, sterile pyrogen-free aqueous and non-aqueous solutions can be used.

The methods and compositions of the presently disclosed subject matter can be used with additional biologically active entities including, but not limited to, cytokines (e.g., IFN-α, IFN-γ, IL-2, IL-4, IL-6, IL-12, TNF, GM-CSF), adjuvants (e.g., complete or incomplete Freund's adjuvant and other art-recognized immunomodulatory adjuvants), anti-tolerance compositions (e.g., antibodies and other compositions directed against regulatory T-cells (Tregs) including, but not limited to anti-CTLA4 antibodies and ONTAK® (Denileukin diftitox, a composition comprising the diphtheria toxin fragments A and B fused to sequences for interleukin-2 (IL-2) that is available from Ligand Pharmaceuticals, Inc., San Diego, Calif., United States of America)), combinations thereof, and/or other immunomodulatory compositions. In accordance with this aspect of the presently disclosed subject matter, the disclosed compositions can be administered in combination therapy with one or more of these biologically active entities.

In some embodiments, the composition further comprises a granulocyte-macrophage colony stimulating factor (GM-CSF) polypeptide or a functional fragment thereof. As used herein, the term “functional fragment” refers to a polypeptide comprising a subsequence of the amino acid sequence of a GM-CSF polypeptide, with the proviso that the polypeptide comprising the subsequence is characterized by having at least a partial immunomodulatory activity of that possessed by a naturally occurring GM-CSF polypeptide. In some embodiments, the immunomodulatory activity is an immunostimulatory activity, and the functional fragment has at least 10%, 20%, 25%, 30%, 40%, 50%, 60%, 70%, 75%, 80%, 90%, 95%, or 100% of the immunostimulatory activity of a wild type GM-CSF polypeptide from the same species as the subject to be treated and/or the in assay by which the activity is tested.

The GM-CSF polypeptide, or the functional fragment thereof, can be present in the composition in any of many forms. In some embodiments, the GM-CSF is present as a recombinant GM-CSF polypeptide. The recombinant GM-CSF polypeptide can be produced by recombinant DNA techniques that are well known in the art (see e.g., Sambrook & Russell, 2001, for a discussion of recombinant polypeptide production). Depending on the species of the subject to which the composition is to be administered, a coding sequence encoding a GM-CSF polypeptide from the appropriate species can be transformed into a cell (e.g., a cell line for the same species) and the recombinant protein purified using standard techniques. Coding sequences for GM-CSFs from several species are publicly available in the GENBANK® database including, but not limited to GENBANK® Accession Nos. NM_{—}000758 (Homo sapiens); XM_{—}527005 (Pan troglodytes); NM_{—}001003245 (Canis familiaris); NM_{—}214118 (Sus scrofa); NM_{—}009969 (Mus musculus); and XM_{—}340799 (Rattus norvegicus). Each of these sequences is incorporated by reference in its entirety, including all annotations present in the webpage of the National Center for Biotechnology Information (NCBI) of the United States of America associated with each, which also includes amino acid sequences encoded thereby.

Thus, in some embodiments the recombinant polypeptide is isolated and purified from the transformed cell. The isolated polypeptide can be added to the composition as is, or in some embodiments, can be encapsulated in a microsphere or liposome. Methods for encapsulating polypeptides in microspheres and/or liposomes are known in the art (see e.g., Hill et al., 2002). Encapsulation methods can be employed in order to provide a sustained immunomodulatory signal relative to providing the GM-CSF polypeptide itself, when such a sustained signal is desired.

Alternatively or in addition, it is also possible to include in the composition a cell that itself expresses a naturally occurring or recombinant GM-CSF polypeptide. As disclosed herein, STO cells have been prepared that express murine GM-CSF. If desired, human fibroblast lines that express a functional human GM-CSF can also be prepared using art-recognized techniques, and these could be included in the compositions (e.g., for use in human subjects).

### II.C. Administration

A composition of the presently disclosed subject matter can be administered to a subject in need thereof in any manner that would be expected to generate an immune response in the subject to at least one antigen produced by the pluripotent cells present within the composition. Suitable methods for administration of a composition of the presently disclosed subject matter include, but are not limited to, intravenous (i.v.), intraperitoneal (i.p.), subcutaneous (s.c.), subdermal (s.d.), intramuscular (i.m.), and/or intratumoral injection, and inhalation.

### II.D. Dose

The presently disclosed subject matter methods comprise administering a therapeutically effective dose of a composition of the presently disclosed subject matter to a subject in need thereof. As defined hereinabove, an “effective amount” is an amount of the composition sufficient to produce a measurable response (e.g., a cytolytic and/or cytotoxic response in a subject being treated). It is understood, however, that the measurable response might not become manifest unless and until the subject develops a tumor or pre-tumor, thereby re-exposing the subject's immune system to an antigen and/or an epitope found on or in a pluripotent cell present in the composition. In some embodiments, the measurable response comprises an activity that inhibits or reduces a rate of tumor growth, or even substantially prevents tumor development and growth.

Actual dosage levels of active ingredients in the compositions of the presently disclosed subject matter can be varied so as to administer an amount of the pluripotent cells that is effective to achieve the desired response for a particular subject. The selected dosage level can depend upon the activity of the composition, the route of administration, combination with other drugs or treatments, the severity of the condition being treated, and the condition and prior medical history of the subject being treated. However, it is within the skill of the art to start doses of the compositions at levels lower than required to achieve the desired effect and to gradually increase the dosage until the desired effect is achieved. In some embodiments of the compositions of the presently disclosed subject matter, the pluripotent cells are present in the composition in an amount ranging from about 1×10⁵ to about 1×10⁶ pluripotent cells per dose. In some embodiments, the pluripotent cells are present in the composition in an amount ranging from about 1×10⁶ to about 1×10⁷ pluripotent cells per dose. In some embodiments, greater than 10⁷ pluripotent cells per dose are present in the composition. It is recognized that this dosage level, which has been shown to be effective in a rodent model, can also be adjusted as necessary for administration to other subjects (including but not limited to subjects of other species) taking into consideration, for example, the size and/or blood volume of the subject. It is within the ability of the skilled artisan in the medical field to extrapolate dosages among different species and among different members of the same species by taking into account these and optionally other parameters.

An additional consideration when administering a composition comprising a pluripotent cell to a subject can be whether the pluripotent cells are capable of proliferating in the subject. This is particularly true with syngeneic pluripotent cells that under certain circumstances can form teratomas when transferred into a subject. In some embodiments, a maximum number of pluripotent cells in the composition are employed, provided that the use of this number of pluripotent cells does not unduly increase the risk that the transferred cells will form a teratoma after administration. In order to minimize the risk of teratoma formation further, the pluripotent cells can be mitotically inactivated (e.g., with mitomycin C, gamma irradiation, etc.) and/or provided as a lysate.

After review of the disclosure herein of the presently disclosed subject matter, one of ordinary skill in the art can tailor the dosages to an individual subject, taking into account the particular formulation, method of administration to be used with the composition, and/or characteristics of the tumor itself, including but not limited to size, growth rate, and number. Further calculations of dose can consider patient height and weight, severity and stage of symptoms, and the presence of additional deleterious physical conditions. Such adjustments or variations, as well as evaluation of when and how to make such adjustments or variations, are known and/or would be apparent to those of ordinary skill in the art upon a review of the instant disclosure.

### III. METHODS OF USE

### III.A. Methods of Prophylaxis

The compositions and methods disclosed herein can be employed for both prophylaxis against as well as treatment for the development of both pre-neoplastic and neoplastic cells.

Thus, in some embodiments the presently disclosed compositions are employed as prophylactic vaccines, and the presently disclosed subject matter provides methods for vaccinating a subject against occurrence of a tumor (i.e., the spontaneous development of a tumor arising from the subject's own cells) in a subject. In some embodiments, the methods comprise administering to a subject in need thereof a composition comprising a plurality of pluripotent cells and one or more pharmaceutically acceptable carriers or excipients.

As used herein, the terms “prophylaxis” and grammatical variants thereof are to be interpreted broadly to encompass not only prevention of the initial occurrence of a tumor and/or a cancer, but also to encompass intermediate levels of prophylaxis including, but not limited to delaying the occurrence and/or re-occurrence of a tumor and/or a cancer in the subject, reducing a rate at which a tumor develops in the subject; and combinations thereof.

In some embodiments, the prophylactic treatments of the presently disclosed subject matter induce in the subject an anti-tumor and/or anti-cancer immune response. In some embodiments, the immune response is sufficient to (a) prevent occurrence of a tumor in the subject; (b) delay occurrence of a tumor in the subject; (c) reduce a rate at which a tumor develops in the subject; (d) prevent recurrence of a tumor in the subject; (e) suppress growth of a tumor in a subject; or (f) combinations thereof. In some embodiments, the immune response comprises a cytotoxic T cell response. In some embodiments, the subject is a human, and in some embodiments the cytotoxic T cell response is mediated by CD8⁺ T cells.

Subjects in need of a prophylactic treatment include subjects that are more likely than the general population to develop a tumor or a cancer. In some embodiments, a human subject in need of prophylactic treatment includes a subject that has a genetic predisposition to developing a certain type of tumor and/or cancer. Genetic bases for disorders of abnormal cellular proliferation have been identified and include, but are not limited to genotypes associated with an increased risk of developing familial adenomatous polyposis (FAP; associated with certain alleles of the APC gene (see Powell et al., 1993)); breast cancer (BRCA1 and 2 genes; Antoniou et al., 2003); and colon cancer (DCC gene; Fearon et al., 1990).

In some embodiments, a human subject in need of prophylactic treatment includes a subject that is predisposed to developing a certain type of tumor and/or cancer as a result of intentional or unintentional exposure to various environmental insults (e.g., cigarette smoking/lung cancer, asbestos exposure/mesothelioma). The methods and compositions disclosed herein can be employed prior to the appearance of any such tumor and/or cancer in an effort to “prime” the immune system of the subject so that the subject's immune system will develop a more robust response to the tumor or cancer, or to an earlier pre-neoplastic precursor cell of the tumor or cancer, than it would have in the absence of the prophylactic treatment.

The nature of the prophylactic treatment as a vaccine is such that the treatment is provided by administering to the subject in need thereof a composition comprising a plurality of pluripotent cells as disclosed herein. As is known in the art, the prophylactic treatment can comprise one administration, or in some embodiments, an initial administration followed by one or more subsequent administrations.

### III.B. Methods of Treatment

The presently disclosed subject matter compositions and methods can also be employed as a part of a treatment regimen for subjects that already have a cancer. Thus, the presently disclosed subject matter also provides methods for preventing the further development and/or proliferation of a cancer.

In some embodiments, the methods disclosed herein comprise administering to a subject in need thereof a composition comprising a plurality of pluripotent cells and one or more pharmaceutically acceptable carriers or excipients. The methods disclosed herein can be employed for treating any tumor and/or cancer in a subject, including but not limited to bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, head tumors, neck tumors, and solid tumors. In some embodiments, the tumor comprises a lung carcinoma.

As is known to those of skill in oncology, combination treatments are frequently employed to treat neoplastic disease. Thus, the presently disclosed compositions and methods can be a part of a broader anti-cancer treatment (i.e., can constitute an adjuvant therapy in combination with other treatments). As such, in some embodiments the presently disclosed methods further comprise providing to the subject an additional anti-tumor therapy such as radiation, chemotherapy, surgical resection of the tumor, or combinations thereof. The additional anti-tumor therapy or combination therapies can be provided to the subject at a time prior to, concurrent with, or subsequent to administering the presently disclosed compositions, and the presently disclosed compositions can be administered at more than one of these time points.

### III.C. Combined Prophylactic/Treatment Methods

In some embodiments, the methods and compositions disclosed herein can be employed for both prophylactic and treatment purposes. An example of a medical condition for which such a combination use would be appropriate would involve the administration to a subject of a composition as disclosed herein to prevent the outgrowth of minimal residual disease (MRD) after the cessation of other shorter term treatments (e.g., surgery, irradiation, and/or chemotherapy). As is known to the medical oncologist, MRD is a significant risk factor for relapse, but is very difficult to detect. Thus, the subject that has concluded his or her cancer treatment is frequently left in doubt as to whether the treatment can be considered a “cure” or just a temporary improvement.

Given that MRD can often not be detected, subjects with MRD are characterized by having no observable tumors and/or cancer. These subjects can then undergo an initial administration of the presently disclosed compositions (or in the case of subjects that have already been administered the presently disclosed compositions, one or more follow-on administrations) in an effort to stimulate the subject's immune system to produce an anti-tumor and/or anti-cancer immune response to address his or her MRD. Employing such a strategy would be expected to minimize the number of more aggressive treatments (e.g., radiation and or chemotherapy) that a subject might require and/or increase the length of time between such treatments.

### IV. METHODS FOR IDENTIFYING AND USING ANTIGENS SHARED BY pluripotent CELLS AND NEOPLASTIC OR PRE-NEOPLASTIC CELLS

The presently disclosed subject matter provides methods for identifying an antigen shared by a pluripotent cell and a neoplastic or pre-neoplastic cell. In some embodiments, the presently disclosed methods comprise (a) administering to a first subject a plurality of pluripotent cells to induce an immune response in the subject against one or more antigens present on one or more of the pluripotent cells; (b) isolating an antiserum, a lymphocyte, a splenocyte, or a combination thereof from the immunized subject; (c) assaying the antiserum, lymphocyte, splenocyte, or combination thereof for an ability to recognize an antigen present on a neoplastic or pre-neoplastic cell; and (d) identifying the antigen present on the neoplastic or pre-neoplastic cell to which the antiserum, lymphocyte, splenocyte, or combination thereof binds.

The presently disclosed subject matter can be employed to screen for antigens that are recognized by the humoral immune system, the cellular immune system, or both the humoral and cellular immune systems. In those embodiments in which the antigens are recognized by the humoral immune system, an antiserum can be isolated from a subject to which the compositions of the presently disclosed subject matter had been administered and tested for the presence of antibodies that recognize antigens expressed by cancer cells.

Once the antiserum is isolated from the subject immunized with the plurality of pluripotent cells, it can be employed to screen a source of potential antigens from neoplastic or pre-neoplastic cells. The neoplastic or pre-neoplastic cell antigens can be supplied for screening in any form. In some embodiments, the neoplastic or pre-neoplastic cell antigens are supplied in the form of a library, including but not limited to an expression library generated from a neoplastic or pre-neoplastic cell of interest. Methods for producing expression libraries are known in the art. See Sambrook & Russell, 2001. Expression libraries can be screened with antisera isolated from subjects immunized with the presently disclosed compositions, and members of the libraries that are bound by antibodies present within the antisera can be identified and subcloned, leading to the identification of the antigen.

As used herein, the term “library” means a collection of molecules. A library can contain a few or a large number of different molecules, varying from at least two molecules to several billion molecules or more. A molecule can comprise a naturally occurring molecule, or a synthetic molecule that is not found in nature. Optionally, a plurality of different libraries can be employed simultaneously for screening.

Antigens that bind to antibodies that are present in an antiserum of a subject to which the presently disclosed compositions has been administered can also be screened by other methods. For example, the serological identification of antigens by recombinant cDNA expression cloning (SEREX; Sahin et al., 1995) method of antigen identification can be employed. Additionally, an antiserum can be used to immunoprecipitate an antigen from source (e.g., a tumor cell or a fraction or component thereof), and the immunoprecipitated antigen can be identified by standard techniques including, but not limited to matrix-assisted laser desorption ionization time-of-flight (MALDI-TOF) mass spectroscopy and sequencing, with our without prior separation on a solid support.

The presently disclosed subject matter can also be employed to screen for antigens that are recognized by the cellular immune system. Assays for screening for induction of cellular immunity are known to those of skill in the art, and include, but are not limited to the technique disclosed in Kawakami et al., 1994, the disclosure of which is incorporated herein in its entirety. Briefly, an individual is immunized with a composition comprising an antigen of choice (e.g., a composition comprising pluripotent cells), and after an appropriate time, splenocytes or lymphocytes are isolated. These cells are then expanded by exposure to the antigen of choice (e.g., by exposure to inactivated pluripotent cells or to a second potential source of antigen such as a cancer cell). cDNA libraries are then transfected into a cell line that expresses MHC proteins that are compatible with the vaccinated individual. The cDNA library-transfected cells are plated in 96 well plates and the cells are expanded. After expansion, a fraction of cells from each well (e.g., one-half) are plated onto another 96 well plate containing expanded CD8+ lymphocytes isolated from the immunized individual. After 24-48 hours, supernatants from the second 96 well plates can be analyzed for interferon gamma (IFN-γ) by Enzyme-linked immunosorbent spot (ELISPOT) assay (Czerkinsky et al., 1983). The cDNAs present in cells from IFN positive duplicate wells can then be recovered (e.g., by PCR) and the process can be repeated with these plasmids until individual cDNAs are identified.

Once an antigen that is shared by a pluripotent cell and a neoplastic or pre-neoplastic cell is identified, the antigen itself and/or an immunogenic fragment thereof can optionally be added to the presently disclosed compositions to supplement the immune response against the antigen. Similarly, an antiserum (e.g., a polyclonal antiserum and/or a monoclonal antiserum) can be generated against the antigen or the immunogenic fragment thereof, and the antiserum can also optionally be added to the presently disclosed compositions to supplement the immune response against the antigen. Techniques for producing polyclonal and monoclonal antisera, as well as antibodies, fragments, and derivatives thereof that can selectively bind to the antigen, are also known.

In some embodiments, the expression library is generated from a neoplastic or pre-neoplastic cell isolated from a subject that has a particular tumor, cancer, or pre-neoplastic condition. In some embodiments, the subject with one or more of these conditions is the subject for whom a treatment with the methods and compositions of the presently disclosed subject matter is desired.

Stated another way, the expression library can be generated from a cell isolated from a subject with a tumor, a cancer, or another like disorder that can thereafter be employed for identifying one or more antigens present on the pluripotent cell that the subject has developed an immune response against, and that is also present on a neoplastic or pre-neoplastic cell that is present within the subject.

Once an antigen that is shared by a pluripotent cell and a neoplastic or pre-neoplastic cell has been identified, this knowledge can be employed to enhance the effectiveness of the compositions and methods described herein. For example, the immune response against antigens that are shared by pluripotent cells and neoplastic or pre-neoplastic cells can be enhanced by adding to the presently disclosed compositions preparations containing the antigen (e.g., a purified antigen or a recombinant antigen), and/or by adding to the presently disclosed compositions antibodies, fragments, or derivatives thereof that selectively bind the antigens. For example, as set forth in EXAMPLE 4, the cytohesin-2 gene product has been identified as an antigen that is shared by pluripotent cells and LLC tumor cells. LLC tumor cells are derived from a lung cancer tumor, and thus anti-cytohesin-2 antibodies can be added to the compositions of the presently disclosed subject matter when the subject to be treated has a cancer (e.g., a lung cancer).

### EXAMPLES

The following Examples have been included to illustrate modes of the presently disclosed subject matter. In light of the present disclosure and the general level of skill in the art, those of skill will appreciate that the following Examples are intended to be exemplary only and that numerous changes, modifications, and alterations can be employed without departing from the scope of the presently disclosed subject matter.

### Example 1

### Spontaneous Carcinogenesis Model

To test whether an ES cell vaccine of the presently disclosed subject matter could protect mice against the initiation and/or proliferation of a spontaneous tumor, a mouse model of chronic pulmonary inflammation following carcinogen treatment that is thought to mimic chronic smoking-associated lung cancer (see Malkinson et al., 1997) was employed.

Six-week-old C57BL/6 mice received antioxidant-free laboratory chow for 2 weeks prior to the carcinogenesis regimen. A single dose of methylcholanthrene (MCA; 15 μg/g mouse weight) dissolved in corn oil was administered i.p. Beginning one week later, mice received six weekly i.p. doses of butylated hydroxytoluene (BHT) dissolved in corn oil. The first dose was 150 μg/g mouse weight, and subsequent doses were 200 μg/g mouse weight. Mice were vaccinated subcutaneously in the left flank, with sterile Hank's balanced salt solution (HBSS) alone, 5×10⁵ murine ES cells alone, or 5×10⁵ murine ES cells+5×10⁵ STO fibroblasts that expressed GM-CSF from an expression vector (STO-GM) at weeks 6, 8, and 10 following initial MCA administration. These formulations were prepared as follows.

For live cell vaccination, the murine embryonic stem (ES) cell line, ES-D3 (American Type Culture Collection (ATCC®); Manassas, Va., United States of America; No. CRL-11632), derived from a 129/Sv mouse, was employed. ES cells were maintained in humidified air with 5% CO₂ in Dulbecco's Modified Eagle's Medium (DMEM) supplemented with 15% ES Cell Qualified fetal bovine serum (GIBCO®, Invitrogen Corporation, Grand Island, N.Y., United States of America), 50 U/mL penicillin, 50 μg streptomycin (GIBCO®, Invitrogen Corporation), 0.1 mM non-essential amino acids (GIBCO®, Invitrogen Corporation), 2 mM L-glutamine (GIBCO®, Invitrogen Corporation) under standard conditions. Leukemia Inhibitory Factor (LIF; CHEMICON® International, Inc., Temecula, Calif., United States of America) was added at a concentration of 80 units/ml (500 pM) to prevent differentiation of the cells during culture. ES cells were periodically evaluated using an anti-stage-specific embryonic antigen-1 (SSEA-1) monoclonal antibody (MC-480; Developmental Studies Hybridoma Bank, Iowa City, Iowa, United States of America) to ensure retention of an undifferentiated state.

Prior to subcutaneous injection, the cells were removed from the plate with enzyme-free cell dissociation solution (Specialty Media, Phillipsburg, N.J., United States of America), washed twice in sterile Hank's buffered salt solution (HBSS), and suspended in HBSS at a concentration of 5×10⁵ cells/ml. The cells (5×10⁵ per inoculation) were administered subcutaneously in the mid-left femoral region of test mice.

In some cases, cells expressing murine GM-CSF (5×10⁵ cells per inoculation) were co-administered with the ES cells, A STO fibroblast cell line (ATCC® No. CRL-1503) was used for this rather than GM-CSF-expressing ES cells because GM-CSF might be expected to promote differentiation of ES cells (Senju et al., 2003) and ES cells are capable of silencing retroviral vectors (Swindle et al., 2004). STO fibroblasts were infected in culture with a replication-defective retrovirus expressing murine GM-CSF (a gift from Dr. Glenn Dranoff, Dana Farber Cancer Institute, Boston, Mass., United States of America), maintained and processed under the same conditions as the ES cells.

Mice were sacrificed 18 weeks after the MCA dose. Tumors were enumerated in fresh lungs inflated at a pressure of 15 cm with 10% buffered formalin. Each lung was examined with a dissection microscope (5× magnification) to obtain the tumor count. The visual scoring scheme was confirmed by histopathological analysis.

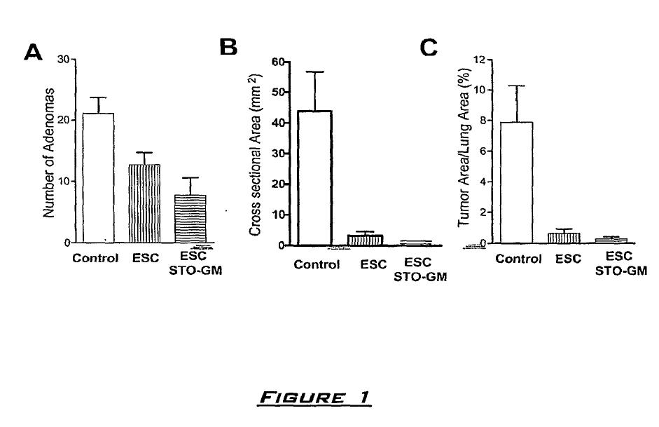

As shown in FIGS. 1A-1C, animals vaccinated with the vaccine regimens were protected against the development of lung cancer in this model. In this regard, there are two notable effects of vaccination. First, vaccinated animals showed a complete lack of large—malignant adenocarcinoma—lung cancers. Second; even if benign adenomas are included in the analysis, the vaccinated animals had total lesion surface areas (expressed as a percentage of total lung surface area) comprising less than 1%, whereas unvaccinated animals had about 10% of total lung volume taken up by tumors.

Immunocytochemistry from these animals showed a striking difference between mock-vaccinated carcinogen-treated mice and those receiving ES cell vaccines. Histologic analysis of complete (i.e., whole lung) sections of two animals from each group 100 μM apart revealed multiple adenocarcinomas (positive for mucin) and small adenomatous lesions in the control unvaccinated mice. In contrast, animals vaccinated with ESC showed either no tumors (1 animal) or rare very small lesions with both low and high grade cytology. Animals vaccinated with ESC/GM-CSF had rare small adenomas (1 animal), with one having rare very small “mixed grade” lesions.

### Example 2

### Analysis of Development of Autoimmune Reaction

Because of the important roles tissue-specific stem cells play in disease resolution in animals, whether there were any noticeable adverse affects of vaccinating healthy adult mice with pluripotent cells was examined, particularly concerning the development of autoimmune antibodies against normal adult stem cells. No overt signs of autoimmune reactions were observed in any of the more than 200 animals vaccinated with ES cells over the course of up to 12 months post vaccination. Particularly, the vaccinated mice at 12 months post vaccination showed no unusual hair loss, skin irritations, or weight loss, showed no overt signs of infection, and had normal appetites. Thus, the vaccinated mice appeared to be as healthy as non-vaccinated mice, which was indicative of there being no significant effect on any stem cell niche resulting from the vaccination with the ES cells. Additionally, immunohistochemical sections of lungs from mice that had received ES cell vaccine displayed no signs of greater tissue damage due to BHT treatment than might be expected if organ-specific stem cells (important in damage repair) had been depleted.

### Example 3

### Analysis of Bone Marrow Stem Cells

The numbers of bone marrow stem cells in three separate groups of three mice each (unvaccinated or vaccinated with either ESC or ESC+STO-GM) were assessed at 20 days following initial vaccination (day 0) and boost (day 10). This was done by real time PCR analyses of the levels of mRNA for three markers of adult bone marrow stem cells (Oct-4, Nanog, and SCL) as previously reported (Ratajczak et al., 2006). A slight but statistically significant decrease in the expression of these markers was observed compared with non-vaccinated mice, suggesting that bone marrow stem cell numbers might have been slightly impacted by the ESC vaccination. However, this slight decrease (˜2 fold reduction in the adult bone marrow stem cell-specific mRNAs) would appear to be negligible compared to the effects on the bone marrow compartment of well known insults to marrow pluripotent cell viability such as irradiation and aging.

### Discussion of EXAMPLES 1-3

The ability of the ES cell vaccination strategies disclosed herein to induce an anti-tumor response to spontaneous tumors was investigated. To determine whether the ES cell vaccination strategy disclosed herein would be successful in preventing the outgrowth of a spontaneous tumor, a mouse model of chronic pulmonary inflammation was employed. Particularly, a mouse model of chronic pulmonary inflammation following carcinogen treatment that is thought to mimic chronic smoking-associated lung cancer (see Malkinson et al., 1997) was chosen. In this model, mice were exposed to a bolus dose of the carcinogen methylcholanthrene (MCA) followed by repetitive administration of butylated hydroxytoluene (BHT). In this model, the carcinogen-induced initiation—combined with tumor promotion arising from chronic pulmonary inflammation caused by a BHT metabolite—predictably leads to numerous lung tumors within four to six months following initiation.

A single dose of MCA was administered i.p., followed by six weekly i.p. doses of BHT. On weeks 6, 8, and 10, mice were vaccinated with nothing (HBSS), ES cells alone or ESC+STO-GM. Animals vaccinated with either ESC or ESC+STO-GM were fully protected against the appearance of large malignant adenocarcinomas but did show similar signs of pulmonary inflammation and some (but significantly fewer) benign adenomas. Thus, it appears that either mode of vaccination with ESC confers complete—or nearly complete—protection against the development of lung cancer in this model.

Accordingly, the presently disclosed compositions and methods represent new approaches to anti-cancer prophylaxis as well as to treatment regimens, either alone or as an adjuvant therapy in addition to or subsequent to conventional oncology treatments (e.g., to treat and/or prevent the outgrowth of minimal residual disease). Summarily, then, the present disclosure provides a broad-spectrum anti-tumor vaccine and/or treatment, wherein the vaccine comprises pluripotent cells in a pharmaceutically acceptable carrier.

### Example 4

### Human ES Cells and Culture Thereof

The human ES cell line BG02 (registered with the National Institutes of Health of the United States; Brimble et al., 2004) was cultured in a fully defined media containing heregulin-1β (an ERBB2/3 ligand), an IGF-I analog, FGF2, and Activin A. This system is known to support robust long-term growth of these and other hESC lines while retaining euploid karyotypes and other properties of pluripotency such as marker expression, differentiation potential, and global transcriptional profile. The defined culture media (DC-HAIF) used herein comprised DMEM/F12, 2 mM GLUTAMAX™, 1× non-essential amino acids, 0.5 U/ml penicillin, 0.5 U/ml streptomycin, 10 μg/ml transferrin (all from Invitrogen Corp., Carlsbad, Calif., United States of America) 0.1 mM β-mercaptoethanol (Sigma, St. Louis, Mo., United States of America), 2% fatty acid-free Cohn's fraction V BSA (Serologicals Corp. Norcross, Ga., United States of America, now part of Millipore Corp., Billerica, Mass., United States of America), 1× Trace Element mixes A, B and C (Cellgro, a part of Mediatech, Inc., Herndon, Va., United States of America), 50 μg/ml Ascorbic Acid (Sigma), 10 ng/ml HRG-1β (Peprotech Inc., Rocky Hill, N.J., United States of America), 10 ng/ml Activin A (R&D systems Inc., Minneapolis, Minn., United States of America), 200 ng/ml LR³—IGF1 (JRH Biosciences, now a part of SAFC Biosciences, Lenexa, Kans., United States of America) and 8 ng/ml FGF2 (R&D Systems). Cultures were maintained on growth factor-depleted MATRIGEL™ (BD Biosciences, Franklin Lakes, N.J., United States of America) diluted 1:200, and were split by disaggregation to single cells using ACCUTASE™ (innovative Cell Technologies, Inc., San Diego, Calif., United States of America).

BG02 cultures were grown to sub-confluence in T25 flasks and harvested by washing with PBS, disaggregating cells with cell dispersal buffer (Invitrogen), and neutralizing with 0.2% BSA. Cells were pelleted by centrifugation and resuspended in culture media. For vaccination studies, 10⁶ cells were injected into each mouse.

### Example 5

### Vaccination with Xenogeneic Pluripotent Cells and Derivatives Thereof

C57BL/6 mice were vaccinated with BG02 human embryonic stem cells (hESCs) provided by Dr. Thomas Schulz (Novocell, Inc., Athens, Ga., United States of America). Cells were grown in serum free defined media until sub-confluent and gently lifted using enzyme free dissociation buffer. The hESCs were resuspended at 1×10⁷ cells/ml in culture medium and 0.1 ml per mouse was injected subcutaneously into the left flanks of a group of 10 mice. In parallel, hESCs were irradiated at 10 gray (Gy) and injected into another group of 10 mice in a similar fashion. Control animals were not injected. 7 days later, the hESC group and irradiated hESC group were boosted with an equivalent dose of either viable hESC or irradiated hESC, respectively.

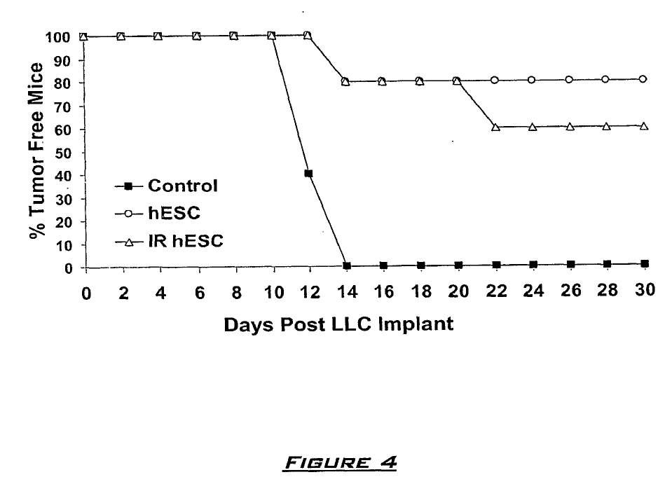

7 days after boost, all mice were challenged subcutaneously on the right flank with 5×10⁴ Lewis Lung Carcinoma (LLC) cells, which are syngeneic to the treated and untreated mice. As shown in FIG. 4, 100% of unvaccinated, control mice developed tumors within 14 days of tumor cell implantation whereas 80% of both hESC vaccinated and irradiated hESC vaccinated mice remained tumor free for 20 days. An additional 20% of the mice vaccinated with irradiated hESC developed small, slow growing tumors at day 20 while the percentage of tumor free mice in the hESC vaccinated group remained at 80%.

### Example 6

### Identification of an Immunoreactive Antigen Expressed in ES and Malignant Lung Cancer Cells

A cDNA expression library was constructed from Lewis Lung Carcinoma (LLC) cells and the serological identification of antigens by recombinant cDNA expression cloning (SEREX; Sahin et al., 1995) method of antigen identification was employed to identify antigens expressed on tumor cells that can be targeted by the immune response elicited by embryonic stem cell (ESC) vaccination. As disclosed herein, ESC vaccination was protective against LLC challenge, and thus immunoscreening a phage expression LLC cDNA library with anti-sera raised against ES cells was expected to yield cross reactive antigens.

Initial, primary screening of an estimated 10,000 cDNAs with anti-sera from ESC vaccinated mice yielded 3 positive clones that were lifted and subjected to a second round of immunoscreening. From the secondary screen, 2 of the original 3 primary clones were identified as false positives while the third yielded two positive subclones. After confirming each subclone by tertiary screening, sequencing revealed that both of the isolated subclones that were identified in the secondary screen were identical to each other. Analysis using the Basic Local Alignment Search Tool (BLAST; available from the website of the National Center for Biotechnology Information (NCBI) of the National Institutes of Health of the United States of America) identified both independently identified cDNAs as corresponding to murine cytohesin-2 (also called pleckstrin homology, Sec7 and coiled-coil domains 2 (Pscd2) or ARF nucleotide-binding-site opener (ARNO); GENBANK® Accession Nos. NM_{—}011181 (nucleotide) and NP_{—}035311 (amino acid)), a membrane bound nucleotide exchange factor for ARF6 GTPase family members. A rudimentary search of the cytohesin-2 sequence against both murine and human EST databases revealed an intriguing expression pattern: embryo>placenta>large variety of tumors>brain>eye>>>adult organs. While not entirely restricted to embryonic and tumor tissue, the pattern of expression indicated significantly higher cytohesin-2 levels in embryos, tumors and, immuno-privileged sites as compared to most normal adult tissues.

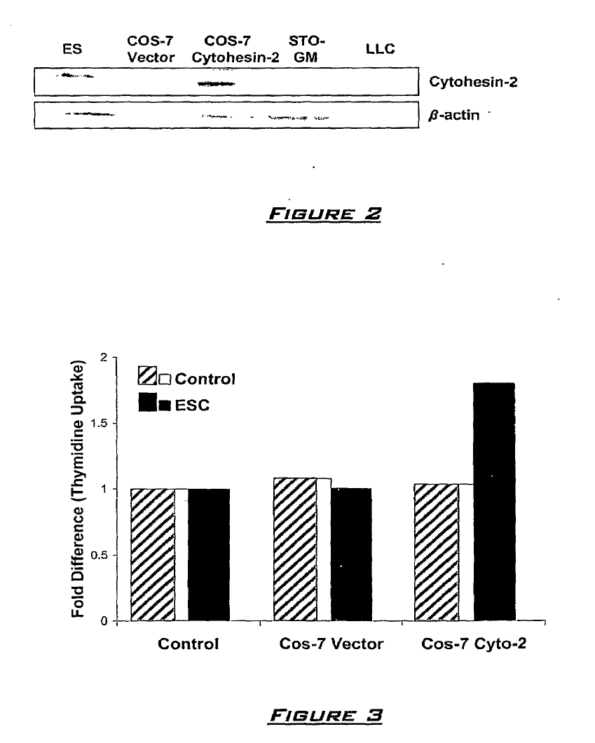

To determine whether cytohesin-2 expression coincided with the whole cell ESC vaccine and the tumor target cells, protein lysates were immunoblotted along with the presently disclosed adjuvant GM-CSF expressing cell line, STO-GM. As shown in FIG. 2, both ES cells and LLC lung cancer target cells expressed detectable levels of cytohesin-2, while the non-transformed STO-GM fibroblasts did not. Additionally, using one of the two cytohesin-2 cDNA expression constructs isolated from subclones immunoreactive with anti-sera raised against ESC, COS-7 cells were transfected and ectopic cytohesin-2 was detectable in these transfectants while vector alone-transfected cell lysates were negative.

### Example 7

### Cytohesin-2 Expression Induced Activation/Proliferation of Splenocytes from ESC-Vaccinated Mice

As disclosed in EXAMPLE 4, SEREX was employed to identify cross reactive antigens that might be indicative of CTL-dependent responses involved in anti-tumor killing induced by ESC vaccination. As such, cytohesin-2 expressed in ES cells might elicit both humoral and CTL responses as a result of ESC vaccination. Since cytohesin-2 is an intracellular protein, ES cell destruction and/or lysis could result in the efficient presentation of cytohesin-2 to immune cells leading to subsequent CTL responses against-tumor cell cytohesin-2.

To investigate whether cytohesin-2 expression was capable of activating splenocytes primed against ES cells, Cos-7 cells were transfected with either vector alone or with a cytohesin-2 expression vector (see FIG. 2). Cell lysates were prepared from each and co-incubated with splenocytes from control and ESC vaccinated mice followed by a short pulse with ³H-thymidine to assess lymphocyte proliferation.

As shown in FIG. 3, lysates from Cos-7 cells transfected with vector alone had no effect on thymidine uptake in control or vaccinated splenocytes, while cytohesin-2 containing lysates selectively induced the activation/proliferation of ESC vaccinated, but not control, splenocytes. These findings were consistent with a potential role for cytohesin-2 as a cross reactive tumor-specific antigen involved in ESC-mediated anti-tumor immune protection.

### Discussion of EXAMPLES 6 and 7

As disclosed herein, cytohesin-2 has been identified as an antigen that is shared by LLC cells and ES cells. A full length recombinant cytohesin-2 was able to activate cell proliferation of splenocytes from ESC vaccinated animals greater than 40-fold. While applicants do not wish to be bound by any particular theory of operation, it is possible that cytohesin-2, possibly in conjunction with one or more other shared ES cell/tumor antigens, contributes to the potent anti-tumor responses that have been observed with ESC vaccination.

A goal of the studies disclosed herein was to further delineate the potential usefulness and clinical viability of a pluripotent cell-based vaccine as an anti-tumor therapeutic and/or prophylaxis agent. In light of the findings presented herein, it is likely that the presently disclosed subject matter can provide: 1) a more accessible and clinically achievable ES cell based vaccine; 2) an ability to evaluate the effectiveness of ES cell vaccination on de novo carcinogen and oncogene-initiated tumorigenesis; and 3) a strategy to identify cross reactive ES cell and tumor antigens that are responsible for ESC anti-tumor immune responses.

The identification of additional immuno-responsive tumor antigens can represent a first step in turning the presently disclosed cell-based vaccine into an even more versatile antigen-based vaccine applicable to most, if not all, clinically relevant human malignancies.

### Example 8

### Reactivity of Antiserum Raised Against Human ES Cells Towards Tumor Antigens

The vaccination experiments disclosed herein suggested there might be substantial antigenic overlap between human embryonic stem cells (hESCs) and LLC cells, a different approach to demonstrate this point would be to identify actual markers that are shared by these cells. Accordingly, monoclonal antibodies (mAbs) were raised against hESCs and tested against LLC cells and MCF-7 cells (a human tumor cell line derived from a mammary tumor). MAbs were generated by immunizing mice with 10⁸ intact hESCs, boosting twice with 10⁷ hESCs, harvesting splenocytes, fusing with a myeloma cell line, and clonal selection of mAb secreting cells.

A panel of 36 mAbs was identified on the basis of immunoreactivity against surface antigens on the cell surface of hESCs but that lacked immunoreactivity with antigens on the surface of differentiated cells. This was determined by examining immunohistochemical staining of in vivo differentiations of hESCs in teratomas. Teratomas contain multiple types of differentiated lineages representing cell types of ectodermal, endodermal, and mesodermal origin. mAbs that were negative on teratomas, or bound to antigens that were tightly restricted in their expression, were identified for further analyses. From within this panel, 16 (44%) of the mAbs immunoreacted with cell surface antigens on LLC cells and on the MCF-7 human mammary tumor cell line (Table 2). An additional antibody immunoreacted with LLC cells but not with MCF7s.

TABLE 2
Immunoreactivity of Monoclonal Antibodies

| Clone | Isotype | hESCs | *Teratomas | MCF7 | LLC |
|---|---|---|---|---|---|
| 1C4 | IgM | 2+ | R | 2+ | 3+ |
| 2C4 | IgG3 | 3+ | R | 2+ | + |
| 2H2 | IgG1 | 3+ | R | 3+ | + |
| 6E8 | IgM | 3+ | Neg | 3+ | + |
| 6H11 | IgG1 | 2+ | R | + | + |
| 12C7 | IgM | 3+ | R | 3+ | 3+ |
| 4A5 | IgM, κ | 3+ | R | 3+ | 3+ |
| 5C7 | IgM, λ | 2+ | Neg | 3+ | 3+ |
| 12A7 | IgM, λ | 3+ | Neg | 3+ | 3+ |
| 13A6 | IgM, λ | 2+ | Neg | 3+ | 3+ |
| 8G2 | IgM, κ | 3+ | Neg | 2+ | 3+ |

hESCs: human embryonic stem cells.
Teratomas: in vivo differentiation of hESCs to generate a wide range of differentiated lineages. hESC-specific markers should be negative in teratomas or highly restricted.
*Neg: negative; R: highly restricted expression.
3+: strong expression; 2+: moderate expression; +: low level expression.
MCF7: human mammary tumor cell line.
LLC: mouse Lewis lung carcinoma cell line.

To further investigate the overlap of these antigens between hESCs and tumor cells, four of these mAbs were used to stain primary tumor punch biopsy samples from primary breast, ovarian, and/or prostate cancers (Table 3). Consistent with the staining observed on cancer cells in vitro, positively stained cells were detected in these primary tumor samples. Typically, small pockets or clusters of cells were identified in a subset of tumors from each tissue.

TABLE 3
Immunoreactivity of Monoclonal Antibodies
Against Primary Human Tumor Samples

| | Positive Primary Tumors | | |
|---|---|---|---|
| Clone | Breast | Ovarian | Prostate |
| 1C4 | 60% | | 0% |
| 2C4 | 37% | 30% | 5.50% |
| 6E8 | 2.50% | 1% | 9.50% |
| 4A5 | 70% | | |

This analysis demonstrated that specific molecular markers of human pluripotent cells were also present on human and mouse cancer cell lines, and in human tumors. These data add strong support to the hypothesis that ES cells and cancer-initiating cells express common “embryonic” antigens that are not generally found on differentiated or adult cell types.

### Example 9

### Vaccination with Partially Differentiated Murine ES Cells

Murine ES-D3 ES cells (American Type Culture Collection (ATCC®); Manassas, Va., United States of America; No. CRL-11632) were maintained as in EXAMPLE 1. LIF was then removed from the culture medium and the cells were grown in culture medium absent LIF for two weeks before vaccinating mice with the then heterogeneous population of cells comprising partially-differentiated ES cell derivatives. LLC cells were thereafter implanted in the vaccinated mice.

At 15 days post-challenge, all non-vaccinated animals (n=10) had obvious tumors, while no vaccinated animals (n=10) had palpable tumors. The cells grown in the absence of LIF for two weeks were actually slightly more effective than the ES-D3 ES cells themselves in protecting LLC-implanted mice against tumor cell growth.

### REFERENCES

The references listed below as well as all references cited in the specification, including patents, patent applications, journal articles, and all database entries (e.g., GENBANK® and/or National Center for Biotechnology Information (NCBI) Accession numbers, including all annotations presented in the various databases associated with the disclosed sequences), are incorporated herein by reference to the extent that they supplement, explain, provide a background for, or teach methodology, techniques, and/or compositions employed herein.
Antoniou et al. (2003) Am J Hum Genet 72:1117-1130.
Bertram & Janik (1980) Cancer Lett 11:63-73.
Brimble et al. (2004) Stem Cells Dev 13:585-597.
Czerkinsky et al. (1983) J Immunol Methods 65:109-121.
Evans et al. (1981) 292 Nature 154-159.
Fearon et al. (1990) Science 247:49-56.
Hill et al. (2002) Cancer Res 62:7254-7263.
Kawakami et al., (1994) Proc Natl Acad Sci USA 91:6458-6462.
Li et al. (2004) Zygote 12:43-48.
Loo & Cotman (1998) in Cell Biology: A Laboratory Handbook (J. E. Celis, ed.) Vol. 1, pages 65-72, Academic Press, San Diego, Calif., United States of America.
Malkinson et al. (1997) Cancer Res 57:2832-2834.
Martin (1981) 78 Proc Natl Acad Sci USA 7634-7638.
McMahon & Bradley (1990) 62 Cell 1073-1085.
Nagy et al. (1990) 110 Development 815-821.
Nagy et al. (2003) Manipulating the Mouse Embryo: A Laboratory Manual (Third Edition), Cold Spring Harbor Laboratories, Cold Spring Harbor, N.Y., United States of America.
PCT International Patent Application Publication WO 99/53021.
Pera & Trounson (2004) 131 Development 5515-5525.
Powell et al. (1993) N Engl J Med 329:1982-1987.
Ratajczak et al. (2006) Leukemia, 20:857-869.
Robertson (1987) Teratocarcinomas and Embryonic Stem Cells: A Practical Approach IRL Press, Washington, D.C., United States of America.
Sahin et al. (1995) Proc Natl Acad Sci USA 92:11810-11813.
Sambrook & Russell (2001) Molecular Cloning: A Laboratory Manual (3rd ed.) Cold Spring Harbor Laboratory Press, Cold Spring Harbor, N.Y., United States of America.
Senju et al. (2003) Blood 101:3501-3508.
Shamblott et al. (1998) Proc Natl Acad Sci USA 95:13726-13731.
Swindle et al. (2004) J Biol Chem 279:34-41.
Thomson et al., (1995) Proc Natl Acad Sci USA 92:7844-7844.
Thomson et al., (1996) Biol Reprod 55:254-259.
Tucker et al., (1997) 25 Nucleic Acids Res 3745-3746.
U.S. Patent Application Publication Nos. 20050266553; 20060030042.
U.S. Pat. Nos. 5,340,740; 5,453,357; 5,453,357; 5,656,479; 5,670,372; 5,670,372; 5,690,926; 5,690,926; 5,830,510; 5,843,780; 6,011,197; 6,090,622; 6,200,806; 6,245,566; 6,271,436; 6,331,406; 6,333,192; 6,800,480; 6,875,607; 6,921,632; and 7,153,684.
Williams et al. (1988) 336 Nature 684-687.

It will be understood that various details of the presently disclosed subject matter can be changed without departing from the scope of the present disclosure. Furthermore, the foregoing description is for the purpose of illustration only, and not for the purpose of limitation.

What is claimed is:

1. A method for suppressing growth of a cancer in a subject, the method comprising administering to the subject a composition comprising a plurality of pluripotent cells and one or more pharmaceutically acceptable carriers or excipients, whereby growth of the cancer in the subject is suppressed.

2. The method of claim 1, wherein the cancer is selected from the group consisting of bladder carcinoma, breast carcinoma, cervical carcinoma, cholangiocarcinoma, colorectal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head tumor, a neck tumor, and a solid tumor.

3. The method of claim 2, wherein the cancer comprises a lung carcinoma.

4. The method of claim 1, wherein the subject is a mammal.

5. The method of claim 4, wherein the mammal is a human.

6. The method of claim 1, wherein the plurality of pluripotent cells are mammalian pluripotent cells.

7. The method of claim 6, wherein the plurality of pluripotent cells comprise human embryonic stem cells, mouse embryonic stem cells, or a combination thereof.

8. The method of claim 1, wherein the plurality of pluripotent cells are allogeneic or xenogeneic to the subject.

9. The method of claim 4, wherein the pluripotent cells are present in the composition in an amount ranging from about 1×10⁵ to about 1×10⁷ pluripotent cells per dose.

10. The method of claim 1, wherein the composition further comprises a biologically active component comprising an adjuvant, a biological response modifier, or a combination thereof.

11. The method of claim 10, wherein the biological response modifier is selected from the group consisting of a cytokine and an anti-tolerance drug.

12. The method of claim 11, wherein the biological response modifier comprises a granulocyte-macrophage colony stimulating factor (GM-CSF), a functional fragment thereof, or a source thereof.

13. The method of claim 12, wherein the GM-CSF or functional fragment thereof is present in the composition in a form selected from the group consisting of a GM-CSF polypeptide, a cell expressing a GM-CSF polypeptide, and an encapsulated GM-CSF polypeptide.

14. The method of claim 13, wherein the functional GM-CSF polypeptide is encapsulated in a microsphere or a liposome.

15. The method of claim 12, wherein the functional GM-CSF comprises a human GM-CSF polypeptide.

16. The method of claim 11, wherein the anti-tolerance drug is selected from the group consisting of an anti-CTLA4 antibody and ONTAK® (denileukin diftitox).

17. The method of claim 1, wherein the administering is performed prior to a diagnosis of a presence of the cancer in the subject, and the composition is in the form of a vaccine.

18. The method of claim 17, wherein the subject in need thereof has at least one risk factor for development of the cancer.

19. The method of claim 18, wherein the risk factor comprises a genetic predisposition for development of the cancer.

20. The method of claim 18, wherein the risk factor comprises exposure to a carcinogen.

21. The method of claim 20, wherein the exposure comprises use of a tobacco product.

22. The method of claim 1, wherein the administering step is performed at least twice.

23. The method of claim 11, further comprising providing to the subject an additional anti-cancer therapy selected from the group consisting of radiation, chemotherapy, surgical resection, immunotherapy, and combinations thereof.

24. The method of claim 23, wherein the additional anti-cancer therapy is provided to the subject at a time prior to, concurrent with, subsequent to, or combinations thereof, the administering step.

25. The method of claim 24, wherein the additional anti-cancer therapy is provided prior to the administering step and the composition is administered as an adjuvant therapy.

26. The method of claim 1, further comprising exposing the plurality of pluripotent cells to a treatment under conditions sufficient to prevent the pluripotent cells from forming a tumor in the subject.

27. The method of claim 26, wherein the exposing step comprises exposing the plurality of pluripotent cells to at least one chemical, to radiation, or to combinations thereof.

28. A method for inducing an anti-tumor immune response in a subject, the method comprising administering to the subject a composition comprising a plurality of pluripotent cells and one or more pharmaceutically acceptable carriers or excipients.

29. The method of claim 28, wherein the anti-tumor immune response is sufficient to:
(a) prevent occurrence of a tumor in the subject;
(b) delay occurrence of a tumor in the subject;
(c) reduce a rate at which a tumor develops in the subject;
(d) prevent recurrence of a tumor in the subject;
(e) suppress growth of a tumor in a subject; or
(f) combinations thereof.

30. The method of claim 29, wherein the anti-tumor immune response comprises a cytotoxic T cell response against an antigen present in or on a cell of the tumor.

31. The method of claim 30, wherein the subject is a human.

32. The method of claim 31, wherein the cytotoxic T cell response is mediated by CD8+ T cells.

33. The method of claim 28, wherein the composition further comprises a granulocyte-macrophage colony stimulating factor (GM-CSF) or a functional fragment thereof, or a source thereof.

34. The method of claim 33, wherein the GM-CSF or functional fragment thereof is present in the composition in a form selected from the group consisting of a functional GM-CSF polypeptide, a cell expressing a functional GM-CSF polypeptide, an encapsulated functional GM-CSF polypeptide, and combinations thereof.

35. The method of claim 34, wherein the functional GM-CSF polypeptide is encapsulated in a microsphere or a liposome.

36. A composition comprising a plurality of pluripotent cells and one or more pharmaceutically acceptable carriers or excipients, wherein the plurality of pluripotent cells are capable of inducing an antitumor immune response when administered to a subject.

37. The composition of claim 36, wherein the composition is in the form of a vaccine.

38. The composition of claim 36, wherein the pluripotent cells are mammalian pluripotent cells.

39. The composition of claim 37, wherein the mammalian pluripotent cells comprise human embryonic stem cells, mouse embryonic stem cells, or a combination thereof.

40. The composition of claim 36, wherein the plurality of pluripotent cells are inactivated to an extent sufficient to prevent the pluripotent cells from producing a tumor when administered to a subject.

41. The composition of claim 36, wherein the pluripotent cells are present in the composition in an amount ranging from about 1×10⁵ to about 1×10⁷ cells per dose.

42. The composition of claim 36, wherein the pluripotent cells are present in the composition in a form selected from the group consisting of whole cells, fractions of cells, cell lysates, and combinations thereof.

43. The composition of claim 36, wherein the composition further comprises an adjuvant, a biological response modifier, or a combination thereof.

44. The composition of claim 43, wherein the biological response modifier comprises a cytokine selected from the group consisting of an interferon alpha (IFN-α), an interferon gamma (IFN-γ), an interleukin 2 (IL-2), an interleukin 4 (IL-4), an interleukin 6 (IL-6), an interleukin 12 (IL-12), a tumor necrosis factor (TNF), and a granulocyte-macrophage colony stimulating factor (GM-CSF), functional fragments thereof, and combinations thereof.

45. The composition of claim 44, wherein the biological response modifier comprises a granulocyte-macrophage colony stimulating factor (GM-CSF), a functional fragment thereof, or a source thereof.

46. The composition of claim 45, wherein the GM-CSF or functional fragment thereof is present in the composition in a form selected from the group consisting of a GM-CSF polypeptide, a cell expressing a GM-CSF polypeptide, and an encapsulated GM-CSF polypeptide.

47. The composition of claim 46, wherein the functional GM-CSF polypeptide is encapsulated in a microsphere or a liposome.

48. The composition of claim 36, wherein the one or more pharmaceutically acceptable carriers or excipients are pharmaceutically acceptable for use in a human.

49. A method for identifying an antigen shared by a pluripotent cell and a neoplastic or pre-neoplastic cell, the method comprising:
(a) administering to a first subject a plurality of pluripotent cells to induce an immune response in the subject against one or more antigens present on one or more of the pluripotent cells;
(b) isolating an antiserum, a lymphocyte, a splenocyte, or a combination thereof from the immunized subject;
(c) assaying the antiserum, lymphocyte, splenocyte, or combination thereof for an ability to recognize an antigen present on a neoplastic or pre-neoplastic cell; and
(d) identifying the antigen present on the neoplastic or pre-neoplastic cell to which the antiserum, lymphocyte, splenocyte, or combination thereof binds.

50. The method of claim 49, wherein the antigen present on the neoplastic or pre-neoplastic cell is provided in the assaying step as a constituent of a library.

51. The method of claim 50, wherein the library comprises an expression library produced from a neoplastic or pre-neoplastic cell.

52. The method of claim 51, wherein the expression library is produced from a neoplastic or pre-neoplastic cell isolated from a second subject, optionally further wherein the first subject and the second subject are the same individual.

53. The method of claim 52, wherein the neoplastic or pre-neoplastic cell is isolated from the second subject as part of a biopsy or resection.

54. The method of claim 49, wherein the plurality of pluripotent cells comprise human embryonic stem cells, murine embryonic stem cells, or combinations thereof.

55. The method of claim 49, wherein the plurality of pluripotent cells are allogeneic or xenogeneic to the first subject, the second subject, or both the first and the second subjects.

56. The method of claim 49, further comprising removing from the antiserum one or more antibodies that bind to a control tissue in the first subject, the second subject, or both the first and the second subjects.
